(19) United States
(12) Patent Application Publication
(10) Pub. No.: US 2025/0256118 A1
(43) Pub. Date: Aug. 14, 2025

(54) DEFIBRILLATOR WITH TWO OR MORE INDEPENDENTLY SELECTABLE, AND INDEPENDENTLY OPERABLE, POWER SOURCES

(71) Applicants: Aniruddha Atre, Pune (IN); Ashish Gawade, Pune (IN)

(72) Inventors: Aniruddha Atre, Pune (IN); Ashish Gawade, Pune (IN)

(21) Appl. No.: 19/195,523

(22) Filed: Apr. 30, 2025

Related U.S. Application Data
(63) Continuation of application No. PCT/IN2023/050996, filed on Oct. 31, 2023.

(30) Foreign Application Priority Data
Oct. 31, 2022 (IN) 202221061842

(51) Int. Cl. A61N 1/39

(52) U.S. Cl. CPC A61N 1/3975

(57) ABSTRACT

A defibrillator with two or more independently selectable, and independently operable, power sources, comprising: two or more power sources (12a, 12b, 12c, 12d) being independently functioning power sources, characterized, in that, an isolation module (ISM) of power switches configured to remove interdependency between said power sources (12a, 12b, 12c, 12d), by isolating each of said power sources (12a, 12b, 12c, 12d) from each other, said isolation module (ISM) being configured with: an intermittent configuration of switches configured to, selectively, connect power signals and/or ground signals to a load circuitry (50) in order to ensure that, at any given instance, only one power source (12a, 12b, 12c, 12d) is actively connected to said load circuitry (50); and a configuration of multi-position switches (SC) wherein said switches being pre-configured to select only a mode of operation correlative to selection of a power source.

### RELATED APPLICATIONS

This application claims priority under 35 U.S.C. § 120 to, and is a continuation of, co-pending International Application PCT/IN2023/050996, filed Oct. 31, 2023 and designating the US, which claims priority to IN application 20/2221061842, filed Oct. 31, 2022, such IN application also being claimed priority to under 35 U.S.C. § 119. These IN and International applications are incorporated by reference herein in their entireties.

### FIELD

This invention relates to the field of biomedical engineering.

Particularly, this invention relates to a defibrillator with two or more independently selectable, and independently operable, power sources.

### BACKGROUND

Current defibrillators have either one source of power supply (i.e., battery only) or a maximum of two sources of power supply (i.e., an AC mains and a battery) which are dependent on each other for defibrillators device to work within regulatory specifications for charging of high voltage capacitor/s.

In short, when using defibrillator on AC mains, it is necessary to have a battery in good health, and having useful life, because it is always the battery that acts as an intermediate/primary energy source to charge the high voltage capacitor/s.

But, as the battery capacity nears the end of its useful life, this dependency becomes an issue as the battery cannot effectively charge the capacitor/s. Moreover, it shows a false voltage reading indicating that it is fully charged when, in fact, it is not (voltage may be reached but coulomb cannot). This condition causes failure to deliver the required energy to a patient via the defibrillator.

A doctor discovers this only when they are attempting a shock, to the patient, via the defibrillator. A sudden cardiac arrest patient has only 10 minutes to survive. If a shock is not delivered within 10 minutes, the patient dies.

There is, therefore, a need to solve the problem of interdependency of battery and AC mains in a defibrillator.

Furthermore, in battery-based defibrillators, a user is not educated about how to check/verify/validate a current status of battery life other than a fuel gauge indication of battery charging. This problem needs to be solved.

Additionally, in battery-based defibrillators, quality of battery power source is also not indicated. This problem needs to be solved.

Furthermore, in AC-mains-based defibrillators, power quality of AC mains is not indicated to its user. It is, generally, assumed that when a defibrillator is plugged into an AC power outlet, it will always deliver a high integrity power signal which is not the case. This problem needs to be solved.

Prior art devices do not provide any information as to how they have functioned over a period of time; this causes devastating blind spots whilst using the defibrillator whose function is only during most critical stages of a patient's life. This problem needs to be solved.

In prior art, only one or two sources are practically present in all defibrillators. Probability of grid failure in rural areas, for a duration of more than 10 minutes, is nearly 100%. Probability of battery failure is also similar in “less frequent usage” settings such as gynecology hospitals or a primary healthcare clinic. Thus, there is a need for a more solid backup. Hence, there is a need for a hand cranked generator, built into a defibrillator.

In prior art, AC mains and battery pack are always connected to each other. So, when using AC mains for delivering shock, it is parallelly connected to a defibrillator load circuit as well as to the battery. This interdependence, as explained above, is problem-causing.

### SUMMARY

An object of the invention is to provide a defibrillator with two or more power sources such that they are not interdependent on each other, in terms of their functioning, and such that they are independently selectable.

Another object of the invention is to provide a defibrillator with two or more power sources such that they are independent of any grid variations.

Yet another object of the invention is to provide a defibrillator with two or more power sources such that they are independent of battery life or its complications.

Still another object of the invention is to provide a defibrillator where ‘quality’ of power source is indicated to a user to allow a user to make an informed choice regarding power source selected in order to power the defibrillator.

An additional object of the invention is to provide a defibrillator which provides information as to how the defibrillator has functioned over a period of time.

Yet an additional object of the invention is to provide a defibrillator, with two or more power sources, which gives a recommendation about which is a more optimum power source to use at a given point of time at point of care.

According to this invention, there is provided a defibrillator with two or more independently selectable, and independently operable, power sources, said defibrillator comprising:
two or more power sources in order to power the defibrillator, each of said power sources being independently functioning power sources, characterized, in that,
an isolation module of power switches being configured to remove interdependency between said power sources, by isolating each of said power sources from each other, said isolation module being configured with:
   an intermittent configuration of switches configured to, selectively, connect power signals and/or ground signals of said power sources to a load circuitry in order to ensure that, at any given instance, only one power source is actively connected to said load circuitry; and
   a configuration of multi-position switches wherein said switches being pre-configured to select only a mode of operation correlative to selection of a power source, said modes being selectable from an AC-DC power source, a hand-cranked generator power source, and a battery supply power source.

In at least an embodiment, said intermittent configuration of switches consisting, essentially, of switches selected from a group of switches including mechanical switches and solid-state isolated switches.

In at least an embodiment, said power sources being selectable from a group of power sources consisting of an AC-DC power source having an AC adaptor to receive AC power supply and converting it to a DC output voltage, a hand-cranked generator that is manually operable, and a battery supply.

In at least an embodiment, said power sources being selectable from a group of power sources consisting of an AC-DC power source having an AC adaptor to receive AC power supply and converting it to a DC output voltage, a hand-cranked generator that is manually operable, and a battery supply, wherein, grounding of said AC-DC adaptor and battery are made common

In at least an embodiment, said power sources being selectable from a group of power sources consisting of an AC-DC power source having an AC adaptor to receive AC power supply and converting it to a DC output voltage, a hand-cranked generator that is manually operable, and a battery supply, wherein,
when said AC-DC power source is used as said power source, it is isolated from said hand-cranked generator and said battery, and only load circuitry receives power from only said AC-DC power source;
when hand-cranked generator is used as said power source, it is isolated from said AC-DC power source and said battery, and only load circuitry receives power from only said hand-cranked generator; and
when battery is used as said power source, it is isolated from said AC-DC power source and said hand-cranked generator, and only load circuitry receives power from only said battery.

In at least an embodiment, a selection of power sources from said power sources being connected to a configuration of multi-position switches comprising:
four input terminals consisting, essentially, of a first input, a second input, a third input, and a fourth input;
two output terminals consisting, essentially, of a first output and a second output;
   wherein, at least one output terminal is, further, arranged to have one neutral or not-connected/open position and two possible connected positions with respect to said four inputs such that,
in one instance, said first output and said second output being connectable to said first input and said third input;
in another instance, said first output and said second output being connectable to said second input and said fourth input;

thereby, disabling any connection between said first output and said second output to said first input and said fourth input, simultaneously; and

thereby, disabling any connection between said first output and said second output to said second input and said third input, simultaneously.

In at least an embodiment, a selection of power sources from said power sources being connected to a configuration of multi-position switches comprising:
four input terminals consisting, essentially, of a first input, a second input, a third input, and a fourth input;
two output terminals consisting, essentially, of a first output and a second output;

wherein, at least a first input terminal and at least a third input terminal are connected to a positive terminal and a negative terminal of an AC-DC power source selectable from said power sources.

In at least an embodiment, a selection of power sources from said power sources being connected to a configuration of multi-position switches comprising:
four input terminals consisting, essentially, of a first input, a second input, a third input, and a fourth input;
two output terminals consisting, essentially, of a first output and a second output;

wherein, at least a second input terminal and at least a fourth input terminal are connected to a positive terminal and a negative terminal of a battery selectable from said power sources.

In at least an embodiment, a selection of power sources from said power sources being connected to a configuration of multi-position switches comprising:
four input terminals consisting, essentially, of a first input, a second input, a third input, and a fourth input;
two output terminals consisting, essentially, of a first output and a second output;

wherein, in order to achieve hand-cranked mode powered by only said hand-cranked generator, said first output and said second output is decoupled from a combination of inputs formed by said first input along with said third input and formed by said second input along with said fourth input.

In at least an embodiment, a selection of power sources from said power sources being connected to a configuration of multi-position switches comprising:
four input terminals consisting, essentially, of a first input, a second input, a third input, and a fourth input;
two output terminals consisting, essentially, of a first output and a second output;

wherein,
said isolation module comprising an additional switch configuration, being a switching mechanism, configured to connect said AC-DC power source to a battery;
simultaneously, said configuration of multi-position switches also, being, connected to said AC-DC power source and said battery;
said switching mechanism and said configuration of multi-position switches being configured with a microcontroller such that when said switching mechanism is closed, an output of said two output terminals, that further feeds downstream circuits of said defibrillator, disconnects;
said switching mechanism and said configuration of multi-position switches being configured with a microcontroller such that when said switching mechanism is open, an output of said two output terminals, that further feeds downstream circuits of said defibrillator, connect.

In at least an embodiment, said isolation module comprising an additional switch configuration, being a switching mechanism, configured with a coupler to connect and disconnect both positive and negative terminals of the battery from the AC-DC power source simultaneously; thereby, isolating the AC mains ground from the battery ground at all times during defibrillator usage.

In at least an embodiment, a selection of power sources from said power sources being connected to a configuration of multi-position switches comprising:
four input terminals consisting, essentially, of a first input, a second input, a third input, and a fourth input;
two output terminals consisting, essentially, of a first output and a second output;

wherein,

said isolation module comprising an additional switch configuration, being a switching mechanism, configured to connect said AC-DC power source to a battery;

said switching mechanism and said configuration of multi-position switches being configured with a microcontroller such that,

if,
said first output and said second output is connected to said battery; and said first output and said second output is not, simultaneously, being used by a display of said defibrillator;

then,
a notification module is activated to send a notification to said microcontroller to change switch from a configuration formed by said second input and said fourth input to a configuration formed by said first output and said third output.

In at least an embodiment, a selection of power sources from said power sources being connected to a configuration of multi-position switches comprising:
four input terminals consisting, essentially, of a first input, a second input, a third input, and a fourth input;
two output terminals consisting, essentially, of a first output and a second output;

wherein,

said isolation module comprising an additional switch configuration, being a switching mechanism, configured to connect said AC-DC power source to a battery;

said switching mechanism and said configuration of multi-position switches being configured with a microcontroller such that,

if,
said first output and said second output is connected to said battery; and said first output and said second output is not used by a display of said defibrillator;

then,
a notification module is activated to send a notification to said microcontroller to change switch from a configuration formed by said second input and said fourth input to a neutral position.

In at least an embodiment, a selection of power sources from said power sources being connected to a configuration of multi-position switches comprising:
four input terminals consisting, essentially, of a first input, a second input, a third input, and a fourth input;
two output terminals consisting, essentially, of a first output and a second output;

wherein,

said isolation module comprising an additional switch configuration, being a switching mechanism, configured to connect said AC-DC power source to a battery;

said switching mechanism and said configuration of multi-position switches being configured with a microcontroller such that,

if,
said first output and said second output is connected to said battery; and said first output and said second output is not used by a display of said defibrillator;

then,
a notification module is activated to send a notification to said microcontroller to change switch from a configuration formed by said second input and said fourth input to a not connected position.

In at least an embodiment, said defibrillator comprising a tracking module configured to poll each and every source of the two or more power sources provided with said defibrillator in order to receive two or more corresponding polled data, each of said polled data being stored in terms of values correlating to time, each of said polled data with respect to time being compared, by means of a comparator, with pre-defined threshold data to determine corresponding two or more outputs, each output being correlating to status of health of the power sources; thereby, allowing a user to choose an appropriate power source for defibrillator operation.

In at least an embodiment, said defibrillator comprising a tracking module configured to poll each and every source of the two or more power sources provided with said defibrillator, said tracking module is further configured to generate a corresponding actuating signal to prompt a user to conserve battery if system is not in use, in that,
a first sensor being configured to sense battery connection to load circuit for monitoring state of the defibrillator (in use or in OFF condition); and
a second sensor is configured to poll analog voltage signals (being one of key performance parameters) of each of the power sources.

In at least an embodiment, said defibrillator comprising a tracking module configured to poll each and every source of the two or more power sources provided with said defibrillator, said tracking module being coupled to a mode selection module in order to allow a user to select mode of use correlative to power supply to be chosen, each mode being selectable from a group of modes associated with a power source selected from a group of power sources consisting of battery mode power source, AC mains mode power source, and hand cranked mode power source.

In at least an embodiment, said defibrillator comprising a tracking module configured to poll each and every source of the two or more power sources provided with said defibrillator,
a. said tracking module being coupled to a mode selection module in order to allow a user to select mode of use correlative to power supply to be chosen;
b. said tracking module being coupled to a notification mechanism to notify selected mode to a user via a notification; and
c. said tracking module being coupled to a battery power conservation module configured to save battery power when said defibrillator is not in use, said battery power conservation module comprising:
   a battery connected to a main load circuit via a first switch, such that, when said first switch is in closed position, battery is connected to a load circuit,
   said battery connected to a display screen via a second switch, such that,
   when said second switch is in ON position, then said battery is connected to said display screen in order to power on said display screen
   when said first switch is in ON position and said second switch is in OFF position, said microcontroller being configured to sense said OFF position of said second switch, a timer module being activated, through an alternate signal, from the microcontroller module, in order to wake up said display screen after a pre-defined threshold of time in order to display a message indicating to the user that the battery is being drained by the load circuit while said display screen is in OFF mode and said defibrillator is not in use.

In at least an embodiment, said defibrillator comprising a tracking module configured to poll each and every source of the two or more power sources provided with said defibrillator,
a. said tracking module being coupled to a mode selection module in order to allow a user to select mode of use correlative to power supply to be chosen;
b. said tracking module being coupled to a notification mechanism to notify selected mode to a user via a notification; and
c. said tracking module being coupled to a battery power conservation module configured to save battery power when said defibrillator is not in use, said battery power conservation module comprising:
   a battery connected to a main load circuit via a first switch, such that, when said first switch is in closed position, battery is connected to a load circuit,
   said battery connected to a display screen via a second switch, such that,
   when said second switch is in ON position, then said battery is connected to said display screen in order to power on said display screen,
   when said first switch is in ON position and said second switch is in OFF position, said microcontroller being configured to sense said OFF position of said second switch, a timer module being activated, through an alternate signal, from the microcontroller module, in order to turn OFF said first switch; thereby, disconnecting said battery from said load circuit.

### BRIEF DESCRIPTION OF THE ACCOMPANYING DRAWINGS

The invention will now be described in relation to the accompanying drawings, in which:

FIG. 1 illustrates a block diagram of the defibrillator of this invention;

FIG. 2 illustrates a first embodiment of a switch configuration which is a sub-module of the isolation module;

FIG. 3 illustrates a second embodiment of a switch configuration which is a sub-module of the isolation module;

FIG. 4 illustrates a battery power conservation logic; and

FIG. 5 illustrates a mode selection module.

### DETAILED DESCRIPTION

According to this invention, there is provided a defibrillator with two or more independently selectable, and independently operable, power sources.

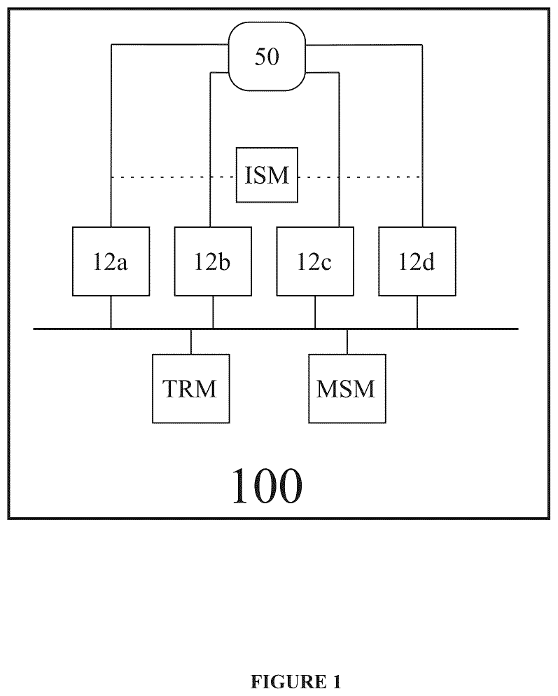

FIG. 1 illustrates a block diagram of the defibrillator (100) of this invention.

In at least an embodiment, two or more power sources (12a, 12b, 12c, 12d) are provided in order to power the defibrillator (100) of this invention. Typically, each of these two or more power sources (12a, 12b, 12c, 12d) are independently functioning power sources so that aging/degradation of one will not affect the performance of the other. This is achieved through a unique arrangement (isolation module (ISM)) of power switches which removes the interdependency.

In at least an embodiment of the isolation module (ISM), independency of sources is achieved by selectively connecting power signals/connections and/or ground signals/connections, of each power source (12a, 12b, 12c, 12d), to a load circuitry (50) through an intermittent configuration of switches (either mechanical or solid state isolated). This ensures that, at any given instance, only one power source is actively connected to the load circuit without shared ground connections. This improves overall power quality also which brings significant improvements in signal processing such as for the ECG portion.

Thus, isolation is achieved;
when AC mains is connected as a power supply, it is only defibrillator output circuit (load circuitry) and not to the battery; and
when battery is connected as power supply, it is isolated from the AC mains and is only defibrillator output circuit (load circuitry) and not to the AC mains;
when on hand cranked mode, it is isolated from both the AC mains and the battery.

This allows relatively for relatively smaller size of AC-DC power supplies, better isolation from AC mains common mode noise which is a problem in prior art.

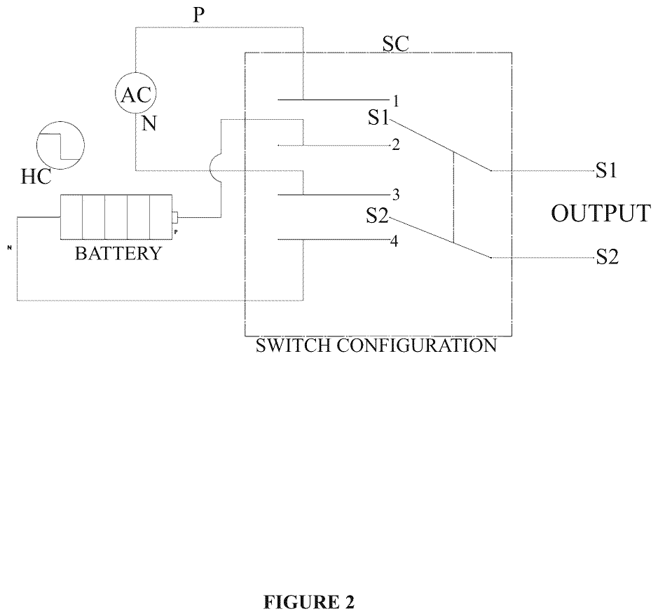

FIG. 2 illustrates a first embodiment of a switch configuration which is a sub-module of the isolation module (ISM).

There are input three power sources available in the system
a. AC-DC: AC adaptor with DC output voltage [power source having an AC adaptor to receive AC power supply and convert it to a DC output voltage];
b. HC: Hand-cranked generator that is manually operated;
c. Battery: Primary battery pack/Secondary battery pack.

Two of the three power sources are connected to a configuration of multi-position switches shown as SC in the FIG. 2. SC contains four input terminals (1, 2, 3, 4) and two output terminals (S1, S2). S1 and S2 are further arranged to have one neutral or not-connected/open position and two possible connected positions with respect to the four inputs (1, 2, 3, 4). So, in one instance, output terminals S1 and S2 can be connected to inputs 1 and 3 and in another instance, output terminals S1 and S2 can be connected to inputs 2 and 4. It is not possible to connect output terminals S1 and S2 to inputs 1 and 4 or to inputs 2 and 3 simultaneously. With this arrangement of switch positions, the input terminals 1 and 3 are connected to a positive terminal and a negative terminal of the AC-DC power supply output. The input terminals 2 and 4 are connected to the positive and negative terminals of the battery pack. A user can, then, choose one of the switch configurations; either switch combination 1-3 or switch combination 2-4 as an input power source. This enables the output terminals (S1-S2) to be selectively connected to the inputs one at a time. It is be noted that ground connections of both the input power sources (AC-C, battery) remain isolated from each other at all times. This is not the case in prior art as ground connections are usually shared between the power sources. This is evident from the fact that, in all defibrillators, when input power source is switched to AC mains, then battery charging and defibrillator output circuit both get connected at the same time. This is possible only when the grounds of AC-DC adaptor and battery are made common. The present invention teaches away from this design scheme and, therefore, isolates ground noises at the source itself due to the disconnection achieved by the configuration of multi-position switches (SC). It saves subsequent task/s, of expensive filtering techniques, to remove AC common mode noise from the signal. When the switch arrangement, from the configuration of multi-position switches (SC), is in neutral mode, that is output S1-S2 is neither connected to 1-3 or 2-4, then the input power source is configured to be defaulted to hand-cranked mode since no other input source (AC-DC, batter, or the like) is available in this configuration.

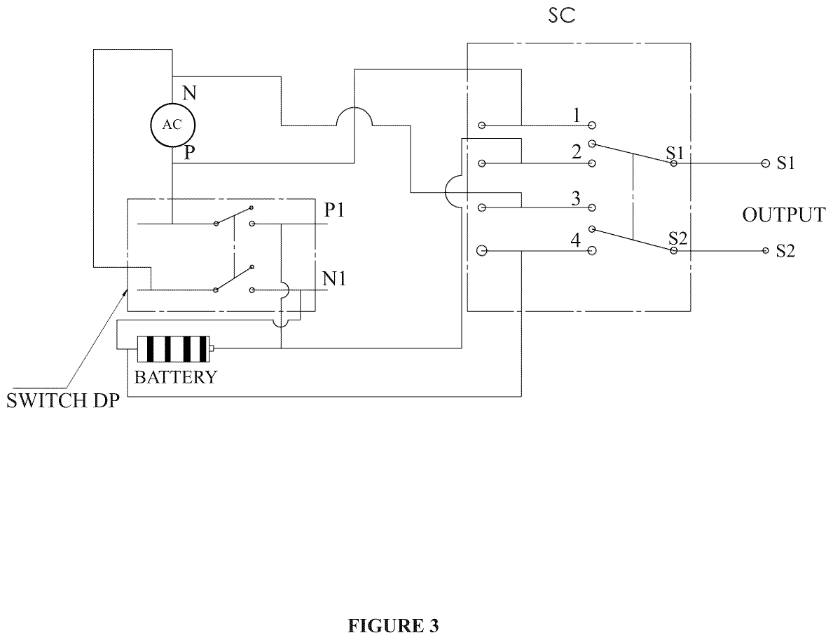

FIG. 3 illustrates a second embodiment of a switch configuration which is a sub-module of the isolation module (ISM).

Another embodiment of the present invention includes an additional switch configuration in the form of a switching mechanism DP as shown in FIG. 3. Through the additional switching mechanism DP, the AC-DC power supply terminals P-N can be connected to battery terminals P1-N1. Simultaneously, configuration of multi-position switches SC is also connected to AC-DC terminals (P-N) and battery terminals (P1-N1) as described in FIG. 2 previously. The switching mechanism (DP) and the configuration of multi-position switches (SC) are configured along with a microcontroller such that when the switching mechanism DP is closed, the output S1-S2 that further feeds downstream circuits is disabled. It is enabled only when the switch DP is kept in open circuit mode. This allows AC-DC power supply to be connected to only one load circuit, at a time, thereby reducing size of power supply required and its cost. In prior arts, the problem of connecting AC-DC power supply to battery and additional load circuit is unsolved.

In yet another embodiment, when the configuration of multi-position switches (SC) switch output (S1-S2) is connected to inputs 2-4 (battery terminals) while the defibrillator screen as driven by output (S1-S2) is turned off by the user, then the microcontroller configured to sense this condition after a predefined time interval, sends an alert to a user in form of an audio-visual alarm to change switch SC from positions 2-4 to either not-connected/neutral position or to position 1-3. This conserves battery energy by reducing parasitic drain on the battery when it is left connected to downstream circuits via output S1-S2. This greatly improves availability of useful energy in the battery for delivery of shocks to patients. This problem is unsolved in prior art as battery is in continuous drain mode as it is always connected to load circuits; thereby, increasing parasitic drain current from the battery.

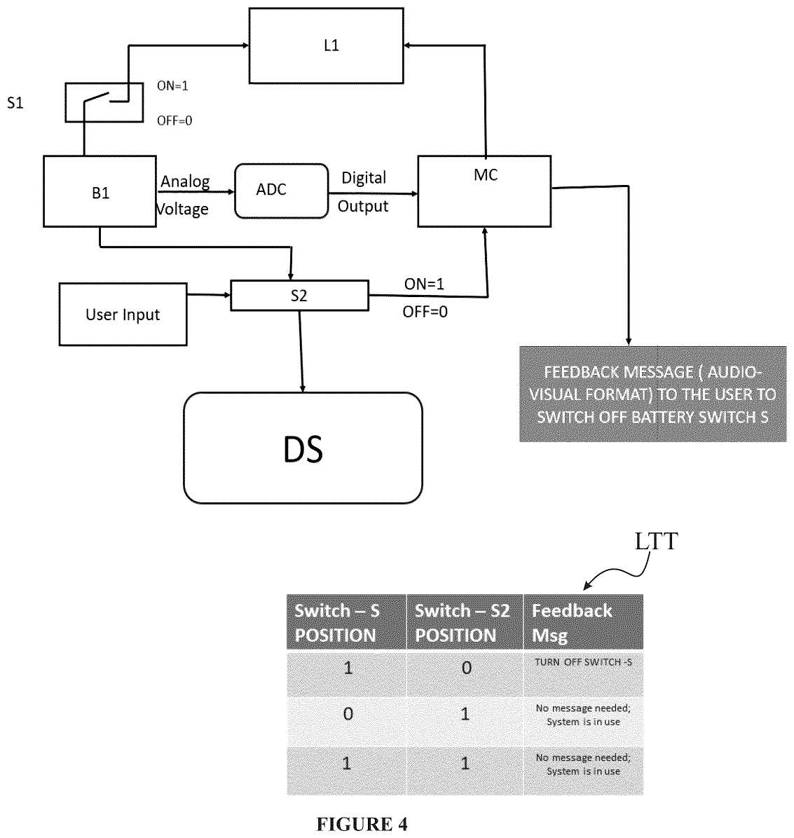

FIG. 4 illustrates a battery power conservation logic.

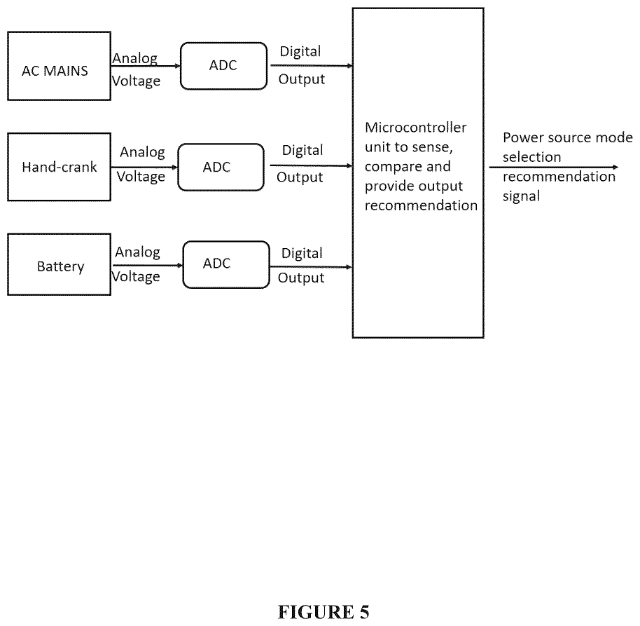

FIG. 5 illustrates a mode selection module.

In at least an embodiment, a tracking module (TRM) is configured to poll each and every source of the two or more power sources (12a, 12b, 12c, 12d) provided with the defibrillator (100) of this invention. Each of this polled data is stored in terms if value correlative to time. Such data, along with analyses, of each of the power sources (12a, 12b, 12c, 12d) provides an output correlative to status of health of each of the power sources (12a, 12b, 12c, 12d); thereby, allowing a user to choose an appropriate power source for defibrillator operation. The analyses are done by collection of data against key performance parameters of each power source, analyzing this through a specified algorithm, and activating a new signal based on the analyses that will help a user to choose a suitable power source for next instance of operation of the device.

In at least an embodiment of the tracking module (TRM), a first sensor is configured to sense battery connection to load circuit for monitoring state of the defibrillator (in use or in OFF condition). The tracking module (TRM) is further configured to generate a corresponding actuating signal to prompt a user to conserve battery if system is not in use.

In at least an embodiment of the tracking module (TRM), a second sensor is configured to poll analog voltage signals (being one of key performance parameters) of each of the power sources (12a, 12b, 12c, 12d); feeding it to a processor for comparing with empirical and statistical data points, along with preset or autogenerated thresholds; thereby, allowing a user to make an ‘informed decision’ regarding selection of power source at the point of care.

In at least an embodiment, a mode selection module (MSM) is provided in order to allow a user to select mode of use correlative to power supply to be chosen. According to preferred embodiments, each mode is correlative to a power source (12a, 12b, 12c, 12d) such as battery mode, AC mains mode, hand cranked mode, along with an additional mode which is a neutral mode. Decision is based on tracking module data. According to preferred embodiments, in order to conserve battery, it is important that users not keep the device on battery mode. They should keep it in “neutral mode” (hand cranked or AC mains mode). Accidentally, if the user keeps it in battery mode, the defibrillator, via a notification mechanism, is configure to notify this erroneous mode to a user via a notification.

As shown in FIG. 4, battery (B1) is connected to main load circuit (L1) via a first switch (S1). The first switch (S1) is a user operated switch. Switch type could be in the form of a mechanical ON-OFF switch or any solid state switch operated by a control signal. Same battery (B1) also powers a display screen of the defibrillator via a second switch (S2). Such a second switch (S2) is also user-operated. This switch type also could be in the form of mechanical switch such as ON-OFF or a rotary dial with ON-OFF positions or any solid state switch operated by a control signal.

When the defibrillator power source is on battery (B1) as described in FIG. 1, above, the first switch (S1) is in closed position and, therefore, battery (B1) is connected to a load circuit (L1). This constitutes default state of the defibrillator when on battery mode. In this state, user has a choice to keep the second switch (S2) in ON or OFF position. If the second switch (S2) is in ON position, then the battery (B) gets connected to the display screen (DS) and that, in turn, gets powered ON. This state is the “in-use” state of the defibrillator wherein the display screen (DS) receives power from the battery and defibrillator is in functional mode.

There is another scenario, wherein the first switch (S1) is in ON position which means that the switch, shown in FIG. 1 of the accompanying drawings, is on battery mode, but the user has kept the second switch (S2) of FIG. 4 in OFF position. This OFF position is sensed by a microcontroller (MC). This state means that the load circuit (L1) is connected to the battery (B1) but the screen is turned OFF; thereby, keeping the defibrillator in OFF state. That is, it is not being used while the battery (B) continues to power load circuit (L1). As soon as this state occurs, a timer module in the microcontroller unit (MC) starts a real time count. As the time count exceeds a pre-defined threshold (preferably, of 30 seconds) while the screen is in OFF mode, then the system, through an alternate signal, from the microcontroller module (MC) wakes up the screen (DS) to display a message indicating to the user that the battery (B) is being drained by the load circuit (L1) while the screen (DS) is in OFF mode and defibrillator is not in use. User is also prompted to turn off the first switch (S1) to OFF position; thereby, disconnecting the battery (B) from the load circuit (L1). Along with a visual message on screen, an audio alarm may also be turned ON to draw attention of the user to this state. This allows the user to conserve battery (B) power when defibrillator is not in use; thereby, prolonging life of the battery.

This logic, described herein, is executed through a logic truth table fed to the microcontroller module firmware. This logic truth table (LTT) is shown in FIG. 4. The Logic truth table (LTT) explains how power conservation logic works for battery conservation when system is not in use.

It may be noted that in other embodiments of this invention, the pre-defined threshold of 30 seconds described above and used by the microcontroller could be any value and all such values are encompassed in the present invention.

With the use of this invention's defibrillator, 100% uptime is expected. Similarly, other renewable energy sources can be highly reliable. E.g., piezo electric device/s which can charge a battery/HV capacitor “on demand (when necessary)”

The TECHNICAL ADVANCEMENT of this invention lies in providing multiple power-sources based defibrillator such that each of the power sources function independently by means of an isolation module; thereby, allowing the defibrillator to function at 100% uptime capacity under all possible power conditions at a critical point of care.

While this detailed description has disclosed certain specific embodiments for illustrative purposes, various modifications will be apparent to those skilled in the art which do not constitute departures from the spirit and scope of the invention as defined in the following claims, and it is to be distinctly understood that the foregoing descriptive matter is to be interpreted merely as illustrative of the invention and not as a limitation.

What is claimed is:

1. A defibrillator with two or more independently selectable and operable power sources, the defibrillator comprising:
two power sources configured to power the defibrillator, wherein the power sources function independently from one another; and
an isolation module isolating each of the power sources from each so as to remove interdependency between the power sources, wherein the isolation module includes,
switches configured to selectively connect power signals and/or ground signals of the power sources to a load circuitry such that, at any given instance, only one power source of the two power sources is connected to the load circuitry, wherein the switches each have at least three different positions that each select one mode of power source operation from modes including AC-DC power, hand-cranked generator power, and battery supply power.

2. The defibrillator of claim 1, wherein the switches are mechanical switches and/or solid-state isolated switches.

3. The defibrillator of claim 1, wherein the power sources are at least one of, an AC-DC power source having an AC adaptor to receive an AC power supply and convert the AC power supply to a DC output voltage, a hand-cranked generator that is manually operable, and a battery supply.

4. The defibrillator of claim 3, wherein the AC-DC adaptor and the battery have a common grounding.

5. The defibrillator of claim 3, wherein,
when the AC-DC power source is used as the power source, it is isolated from the hand-cranked generator and the battery, and the load circuitry receives power from only the AC-DC power source,
when the hand-cranked generator is used as the power source, it is isolated from the AC-DC power source and the battery, and the load circuitry receives power from only the hand-cranked generator, and
when battery is used as the power source, it is isolated from the AC-DC power source and the hand-cranked generator, and the load circuitry receives power from only the battery.

6. The defibrillator of claim 1, wherein the switches include,
four input terminals including a first input, a second input, a third input, and a fourth input, and
two output terminals including a first output and a second output, wherein at least one of the output terminals is configured to have one neutral position and two possible connected positions of the four inputs such that the first output and the second output are connected either to the first input and the third input, or to the second input and the fourth input, such that a connection between the first output and the second output is not connected to the first input and the fourth input simultaneously, and such that a connection between the first output and the second output is not connected to the second input and the third input simultaneously.

7. The defibrillator of claim 1, wherein the switches include,
four input terminals including a first input, a second input, a third input, and a fourth input, and
two output terminals including a first output and a second output, wherein, at least a first input terminal and at least a third input terminal are connected to a positive terminal and a negative terminal of an AC-DC power source selectable from the power sources.

8. The defibrillator of claim 1, wherein the switches include,
four input terminals consisting, essentially, of a first input, a second input, a third input, and a fourth input, and
two output terminals including a first output and a second output, wherein, at least the second input terminal and at least the fourth input terminal are connected to a positive terminal and a negative terminal of a battery of the power sources.

9. The defibrillator of claim 1, wherein the switches include,
four input terminals including a first input, a second input, a third input, and a fourth input, and
two output terminals including a first output and a second output, wherein, to achieve hand-cranked mode powered by only the hand-cranked generator, the first output and the second output are decoupled from a combination of inputs formed by the first input and the third input and formed by the second input and the fourth input.

10. The defibrillator of claim 1, wherein the switches include,
four input terminals including a first input, a second input, a third input, and a fourth input, and
two output terminals including a first output and a second output, wherein the isolation module includes an additional switch configured to connect the AC-DC power source to a battery, while simultaneously the switches are connected to the AC-DC power source and the battery; the defibrillator further comprising:
a microcontroller configured to control the switches such that when closed, an output of the two output terminals that further feeds downstream circuits of the defibrillator disconnects and such that when the switching mechanism is open, an output of the two output terminals that further feeds downstream circuits of the defibrillator connects.

11. The defibrillator of claim 1, wherein the isolation module includes a coupler configured to connect and disconnect both positive and negative terminals of the battery from the AC-DC power source simultaneously so as to isolate the ground of na AC main from the battery ground when the defibrillator is in use.

12. The defibrillator of claim 1, wherein the switches include,
four input terminals including a first input, a second input, a third input, and a fourth input, and
two including a first output and a second output, wherein the isolation module includes a switching mechanism configured to connect the AC-DC power source to a battery, and wherein the switching mechanism and the switches are configured with a microcontroller such that if the first output and the second output is connected to the battery, and the first output and the second output is not, simultaneously, being used by a display of the defibrillator, then a notification module is activated to send a notification to the microcontroller to change switch from a configuration formed by the second input and the fourth input to a configuration formed by the first output and the third output.

13. The defibrillator of claim 1, wherein the switches include,
four input terminals including a first input, a second input, a third input, and a fourth input, and
two output terminals including a first output and a second output, wherein the isolation module includes an additional switching mechanism configured to connect the AC-DC power source to a battery, and wherein the additional switching mechanism and the switches are configured with a microcontroller such that, if the first output and the second output are connected to the battery, and the first output and the second output is not used by a display of the defibrillator, then a notification module is activated to send a notification to the microcontroller to change switch from a configuration formed by the second input and the fourth input to a neutral position.

14. The defibrillator of claim 1, wherein the switches include,
four input terminals including a first input, a second input, a third input, and a fourth input,
two output terminals including a first output and a second output, wherein the isolation module includes a switching mechanism configured to connect the AC-DC power source to a battery, and
a microcontroller configured with the switching mechanism and the switches such that if the first output and the second output are connected to the battery, and the first output and the second output are not used by a display of the defibrillator, then a notification module is activated to send a notification to the microcontroller to change a switch from a configuration formed by the second input and the fourth input to a not connected position.

15. The defibrillator of claim 1, further comprising:
a tracking module configured to poll each of the power sources to receive two or more corresponding polled data, each of the polled data being stored in terms of values correlating to time, each of the polled data with respect to time being compared, by a comparator, with a threshold data to determine corresponding two or more outputs, each output correlating to status of health of the power sources so as to allow a user to choose an appropriate power source for defibrillator operation.

16. The defibrillator of claim 1, further comprising:
a tracking module configured to poll each of the power sources and to generate a corresponding actuating signal to prompt a user to conserve battery if system is not in use, wherein the tracking module includes a first sensor configured to sense battery connection to a load circuit for monitoring state of the defibrillator and a second sensor configured to poll analog voltage signals of each of the power sources.

17. The defibrillator of claim 1, further comprising:
a tracking module configured to poll each of the power sources, wherein the tracking module is coupled to a mode selection module to allow a user to select one of the modes of use correlative to a power supply to be chosen.

18. The defibrillator of claim 17, further comprising:
a notification mechanism coupled to the tracking module to notify the selected mode to a user; and
a battery power conservation module coupled with the tracking module to save battery power when the defibrillator is not in use, wherein the battery power conservation module includes,
a battery connected to a main load circuit via a first switch such that, when the first switch is in closed position, battery is connected to a load circuit, and
a display screen connected to the battery via a second switch such that, when the second switch is in ON position, then the battery is connected to the display screen to power on the display screen, and when the first switch is in ON position and the second switch is in OFF position the display screen is configured to wake up after a threshold of time to display a message indicating to the user that the battery is being drained by the load circuit while the display screen is in OFF mode and the defibrillator is not in use.

19. The defibrillator of claim 1, further comprising:
a tracking module configured to poll each of the two power sources, wherein the tracking module is coupled to a mode selection module to allow a user to select mode of use correlative to power supply to be chosen;
a notification mechanism couples to the tracking module to notify selected mode to a user via a notification; and
a battery power conservation module configured to save battery power when the defibrillator is not in use, wherein the battery power conservation module includes,
a battery connected to a main load circuit via a first switch such that, when the first switch is in closed position, the battery is connected to a load circuit, and
a display screen connect to the battery via a second switch such that when the second switch is in an on position, then the battery is connected to the display screen to power on the display screen.
